(19) United States
(12) Patent Application Publication
(10) Pub. No.: US 2024/0424041 A1
(43) Pub. Date: Dec. 26, 2024

(54) FLAMMULINA VELUTIPES PROTEOGLYCAN (FPG) AND DUAL-WAVELENGTH SCREENING METHOD AND USE THEREOF

(71) Applicant: NANJING UNIVERSITY OF FINANCE & ECONOMICS, Nanjing (CN)

(72) Inventors: Wenjian YANG, Nanjing (CN); Ye HU, Nanjing (CN); Qiuhui HU, Nanjing (CN); Anxiang SU, Nanjing (CN); Minhao XIE, Nanjing (CN); Fei PEI, Nanjing (CN); Jianhui LIU, Nanjing (CN); Hui XU, Nanjing (CN)

(21) Appl. No.: 18/632,657

(22) Filed: Apr. 11, 2024

(30) Foreign Application Priority Data
Jun. 25, 2023 (CN) 202310745157.6

(51) Int. Cl. C07K 14/375; A61K 9/28; A61K 38/00; A61P 35/00; C07K 1/36; G01N 30/06; G01N 30/74

(52) U.S. Cl. CPC A61K 36/06; A61K 9/2813; A61K 9/2826; A61K 9/286; A61P 35/00; C07K 14/375; G01N 30/74; A61K 36/07; A61K 38/00; C07K 1/36; G01N 2030/062

(57) ABSTRACT

The present disclosure provides a Flammulina velutipes proteoglycan (FPG) and a dual-wavelength screening method and use thereof, and relates to the technical field of biomedicine. In the present disclosure, components such as polysaccharides, proteoglycan (PG), and free proteins in the Flammulina velutipes are extracted through water extraction. The free proteins are removed by protein removal, and the polysaccharides and PG are separated by alcohol precipitation. Sepharose FF ion exchange chromatography and Sephadex G-200 chromatography separation are conducted with water as an eluent. Dual-wavelength detection is conducted to collect an eluate with absorption peaks at both 480 nm and 295 nm wavelengths, such that a component—the FPG, which has a protein structure and a polysaccharide structure, is retained.

### CROSS-REFERENCE TO RELATED APPLICATION

This patent application claims the benefit and priority of Chinese Patent Application No. 202310745157.6 filed with the China National Intellectual Property Administration on Jun. 25, 2023, the disclosure of which is incorporated by reference herein in its entirety as part of the present application.

### TECHNICAL FIELD

The present disclosure belongs to the technical field of biomedicine, and particularly relates to a Flammulina velutipes proteoglycan (FPG) and a dual-wavelength screening method and use thereof, and an anti-liver cancer drug, an FPG composite particle, and an FPG composite lozenge.

### BACKGROUND

Flammulina velutipes proteoglycan (FPG) is a high-molecular polymer in which polysaccharide chains and protein (peptide) chains are linked together by covalent bonds. Proteoglycans (PGs) have both the physical and chemical properties of polysaccharides (such as immune regulation, anti-tumor function, and memory improvement) and the physical and chemical properties of proteins (peptides). At present, the FPG is mostly isolated and purified according to a preparation method of the Flammulina velutipes polysaccharide (FVP). A mixture of polysaccharides, proteins, PG and other substances is obtained through hot water extraction. Protein removal is conducted by a Sevag method and then chromatographic separation is conducted, and a sample solution is collected according to a protein absorption peak, and then concentrated and freeze-dried. However, most of the PG products obtained by existing FPG separation methods are polysaccharides, and there is a poor purity of FPG (less than 60%).

The prior art “Isolation, characterization and HepG-2 inhibition of a novel proteoglycan from Flammulina velutipes. International Journal of Biological Macromolecules 189 (2021) 11-17” has disclosed a preparation method of a novel proteoglycan PGD1-1, and the prepared proteoglycan PGD1-1 has a purity of 87.21%. However, the proteoglycan PGD1-1 has an inhibitory activity on HepG-2 cells of only 37.96%, indicating that there is an insufficient inhibitory activity on the HepG-2 cells.

### SUMMARY

In view of this, an objective of the present disclosure is to provide an FPG and a dual-wavelength screening method and use thereof, and an anti-liver cancer drug, an FPG composite particle, and an FPG composite lozenge. The FPG obtained by the dual-wavelength screening method has a purity of not less than 90% and shows a high inhibitory activity against liver cancer.

To achieve the above objective, the present disclosure provides the following technical solutions:

The present disclosure provides a dual-wavelength screening method of an FPG, including the following steps:
subjecting Flammulina velutipes to water extraction and protein removal in sequence to obtain a protein-free water extract;
mixing the protein-free water extract with absolute ethanol to allow alcohol precipitation to obtain a proteoglycan (PG) precipitate;
dialyzing the PG precipitate to obtain a crude PG; and
subjecting the crude PG to separation by Sepharose FF ion exchange chromatography and Sephadex G-200 chromatography in sequence with water as an eluent, and collecting a component with absorption peaks at both 480 nm and 295 nm to obtain the FPG.

Preferably, a dry weight of the Flammulina velutipes and a volume of water for the water extraction are at a ratio of 1 g:20 mL to 1 g:30 mL; and
the water extraction is conducted at 85° C. to 95° C. for 3.5 h to 4.5 h.

Preferably, the protein-free water extract and the absolute ethanol are at a volume ratio of 1:3.5 to 1:4; and
the alcohol precipitation is conducted at 0° C. to 25° C. for 24 h to 72 h.

Preferably, the dialyzing is conducted at a molecular weight cut-off of 3 kDa to 5 kDa for 20 h to 28 h.

Preferably, the crude PG is dissolved in water to allow the separation by the Sepharose FF ion exchange chromatography; and
a dry weight of the crude PG and a volume of the water are at a ratio of 1 g:1 to 1 g:15 mL.

The present disclosure further provides an FPG prepared by the dual-wavelength screening method, where a monosaccharide unit of the FPG includes glucose, D-galactose, and xylose; and a peptide chain end of the FPG is ligated to a polysaccharide chain by an O-glycosidic bond.

The present disclosure further provides use of the FPG in preparation of a functional food or an anti-liver cancer drug.

The present disclosure further provides an anti-liver cancer drug, including the FPG and a pharmaceutically acceptable excipients.

The present disclosure further provides an FPG composite particle, including the following components in parts by mass: 65 parts to 85 parts of the FPG, 5 parts to 15 parts of maltodextrin, 5 parts to 10 parts of fructooligosaccharide, 3 parts to 8 parts of erythritol, 5 parts to 8 parts of calcium carbonate, and 2 parts to 5 parts of a binder.

The present disclosure further provides an FPG composite lozenge, including a core tablet and a coating layer coated on a surface of the core tablet; where

the core tablet includes the following components in parts by mass: 65 parts to 85 parts of the FPG, 5 parts to 15 parts of maltodextrin, 5 parts to 10 parts of fructooligosaccharide, 3 parts to 8 parts of erythritol, 5 parts to 8 parts of calcium carbonate, and 2 parts to 5 parts of a binder.

The present disclosure provides a dual-wavelength screening method of an FPG. In the present disclosure, components such as polysaccharides, PG, and free proteins in the Flammulina velutipes are extracted through water extraction. The free proteins are removed by protein removal, and the polysaccharides and PG are separated by alcohol precipitation. Sepharose FF ion exchange chromatography and Sephadex G-200 chromatography separation each are conducted with water as an eluent. Dual-wavelength detection is conducted to collect an eluate with absorption peaks at both 480 nm and 285 nm wavelengths, such that a component—the FPG, which has a protein structure and a polysaccharide structure, is retained. The FPG has high purity and yield; and the dual-wavelength screening method is simple in process and operations with low cost, and environmental friendliness, which is suitable for industrial production. As shown in the test results of the examples, the FPG prepared by the present disclosure has a purity of not less than 90% and a yield of not less than 75%.

The present disclosure further provides an FPG prepared by the dual-wavelength screening method, where a monosaccharide unit of the FPG includes glucose, D-galactose, and xylose; and a peptide chain end of the FPG is ligated to a polysaccharide chain by an O-glycosidic bond. The FPG has high purity, absorption wavelength of polysaccharides and proteins, and anti-liver cancer activity. Therefore, the FPG exhibits desirable application prospects in preparation of anti-liver cancer drugs and functional foods (such as FPG composite lozenge and FPG composite particle). As shown in the test results of the examples, 200 μg/mL FPG solution has an inhibition rate of not less than 41% on HepG-2 cells.

### BRIEF DESCRIPTION OF THE DRAWINGS

FIG. 1 shows an infrared spectrum of the FPG prepared in Example 1; and

FIG. 2 shows a physical map of the FPG prepared in Example 1.

### DETAILED DESCRIPTION OF THE EMBODIMENTS

The present disclosure provides a dual-wavelength screening method of an FPG, including the following steps:
subjecting Flammulina velutipes to water extraction and protein removal in sequence to obtain a protein-free water extract;
mixing the protein-free water extract with absolute ethanol to allow alcohol precipitation to obtain a proteoglycan (PG) precipitate;
dialyzing the PG precipitate to obtain a crude PG; and
subjecting the crude PG to separation by Sepharose FF ion exchange chromatography and Sephadex G-200 chromatography in sequence with water as an eluent, and collecting a component with absorption peaks at both 480 nm and 295 nm to obtain the FPG.

Unless otherwise specified, the raw materials used in the present disclosure are all commercially available commodities.

In the present disclosure, Flammulina velutipes is subjected to water extraction and protein removal in sequence to obtain a protein-free water extract. The water extraction and protein removal of the Flammulina velutipes preferably includes: mixing the Flammulina velutipes with water to allow the water extraction to obtain a water extract; and subjecting the water extract to the protein removal to obtain a protein-free water extract. The Flammulina velutipes is preferably a Flammulina velutipes dry powder, and the Flammulina velutipes dry powder has a particle size of preferably no more than 250 μm. A dry weight of the Flammulina velutipes (that is, a mass of the Flammulina velutipes dry powder) and a volume of the water are at a ratio of preferably 1 g:20 to 1 g:30 mL, more preferably 1 g:22 to 1 g:28 mL, and even more preferably 1 g:24 to 1 g:25 mL. The water extraction is conducted at preferably 85° C. to 95° C., more preferably 90° C. for preferably 3.5 h to 4.5 h, more preferably 4 h. The protein removal is conducted preferably by Sevag method, and a reagent for the protein removal by Sevag method is preferably a Sevag reagent (a mixture of chloroform and n-butanol with a ratio of 5:1 by volume). The water extract and the Sevag reagent are at a volume ratio of preferably 3-:1 to 4:1, more preferably 3.5:1 to 4:1.

In the present disclosure, the protein-free water extract is mixed with absolute ethanol to allow alcohol precipitation to obtain a PG precipitate. The protein-free water extract and the absolute ethanol are at a volume ratio of preferably 1:3.5 to 1:4, more preferably 1:3.6 to 1:3.9, even more preferably 1:3.7 to 1:3.8. The alcohol precipitation is conducted at preferably 0° C. to 25° C., more preferably 10° C. to 15° C. for preferably 24 h to 72 h, more preferably 48 h to 72 h.

In the present disclosure, after obtaining the PG precipitate, the PG precipitate is dialyzed (referred to as first dialysis) to obtain a crude PG. The first dialysis is preferably conducted using a dialysis bag, and the dialysis bag has a pore size of preferably 3 kDa to 5 kDa, more preferably 4 kDa; the first dialysis is conducted at preferably 10° C. to 30° C., more preferably 20° C. to 30° C. for preferably 20 h to 28 h, more preferably 22 h to 26 h. The first dialysis can remove small molecular substances such as salts and retain components such as proteins and polysaccharides. After the first dialysis is completed, a dialyzed crude PG is preferably dried to obtain the crude PG. The drying is preferably freeze-drying, and the freeze-drying is conducted at preferably-60° C. to −40° C., more preferably −60° C. to −50° C. for preferably 48 h to 96 h, more preferably 60 h to 72 h.

In the present disclosure, the crude PG is subjected to separation by Sepharose FF ion exchange chromatography and Sephadex G-200 chromatography in sequence with water as an eluent, and a component with absorption peaks at both 480 nm and 295 nm is collected to obtain the FPG. An eluent used in the Sepharose FF ion exchange chromatography (i.e. DEAE Sepharose Fast Flow) separation and the Sephadex G-200 chromatography separation is water, preferably ultrapure water; and the water has a flow rate of preferably 0.5-2 mL/min, more preferably 1-1.5 mL/min. The crude PG is preferably dissolved in water to allow the separation by the Sepharose FF ion exchange chromatography; and a dry weight of the crude PG and a volume of the water are at a ratio of preferably 1 g:1 to 1 g:15 mL, more preferably 1 g:2 to 1 g:10 mL, and even more preferably 1 g:3 to 1 g:5 mL. The Sephadex G-200 chromatography has a separation range of preferably 5 kDa to 600 kDa. After the collection, the collected eluate is preferably dialyzed (referred to as second dialysis) and then dried to obtain the FPG. The conditions for the second dialysis are preferably the same as those for the first dialysis, and will not be described again; the second dialysis removes small-molecular salts and the like to further improve a purity of the FPG. The drying is preferably freeze-drying, and the freeze-drying is conducted at preferably −60° C. to −40° C., more preferably-60° C. to −50° C. for preferably 48 h to 96 h, more preferably 60 h to 72 h.

The present disclosure further provides an FPG prepared by the dual-wavelength screening method, where a monosaccharide unit of the FPG includes glucose, D-galactose, and xylose; and a peptide chain end of the FPG is ligated to a polysaccharide chain by an O-glycosidic bond. In the present disclosure, the glucose, the D-galactose, and the xylose are at a molar ratio of preferably (24-25):(2-4):(1-6), more preferably 24:4:1 or 25:2:6. The FPG has a molecular weight of preferably 30 kDa to 33.5 kDa, more preferably 33.05 kDa or 30.42 kDa. The FPG includes sugar, protein, and uronic acid. In the FPG, the sugar is preferably 93.38 wt %, the protein is preferably 2.33 wt %, and the uronic acid is preferably 1.53 wt %.

The present disclosure further provides use of the FPG in preparation of a functional food or an anti-liver cancer drug. The FPG in the functional food is added at preferably 40 wt % to 85 wt %, more preferably 65 wt % to 85 wt %. The FPG in the anti-liver cancer drug is added at preferably 60 wt % to 95 wt %, more preferably 80 wt % to 95 wt %.

The present disclosure further provides an anti-liver cancer drug, including the FPG and a pharmaceutically acceptable excipients. In the present disclosure, there are no special limitations on the pharmaceutically acceptable excipients, and excipients well known to those skilled in the art can be used. There is no particular limitation on a dosage form of the anti-liver cancer drug, and dosage forms well known to those skilled in the art can be used.

The present disclosure further provides an FPG composite particle, including the following components in parts by mass: 65 parts to 85 parts, preferably 70 parts to 80 parts, and more preferably 75 parts of the FPG; 5 parts to 15 parts, preferably 8 parts to 12 parts, and more preferably 10 parts of maltodextrin; 5 parts to 10 parts, preferably 6 parts to 9 parts, and more preferably 7 parts to 8 parts of fructooligosaccharide; 3 parts to 8 parts, preferably 4 parts to 7 parts, and more preferably 5 parts to 6 parts of erythritol; 5 parts to 8 parts, preferably 5.5 parts to 7.5 parts, and more preferably 6 parts to 7 parts of calcium carbonate; and 2 parts to 5 parts, preferably 2.5 parts to 4.5 parts, and more preferably 3 parts to 4 parts of a binder. The binder preferably includes hydroxypropyl methylcellulose (HPMC) and/or gum arabic. The FPG composite particle is preferably taken orally, with a dosage of preferably 1.2 to 1.8 g/day.

In the present disclosure, a preparation method of the FPG composite particle preferably includes the following steps: mixing the FPG, maltodextrin, fructooligosaccharide, erythritol, calcium carbonate, HPMC, and water to allow granulation and drying to obtain the FPG composite particle; and compressing the FPG composite particle into tablets to obtain a health-care candy lozenge. In the present disclosure, the raw materials for preparing the FPG lozenge are preferably superfine grinded and then screened before use. There is no particular limitation on the superfine grinding herein, as long as the raw materials can be grinded to a mesh size of no less than 300 mesh. A sieve mesh has a size of preferably 300 mesh, and a resulting undersize is collected. In the present disclosure, the granulation is preferably conducted in a boiling granulator or a spray granulator. The health candy particles have a particle size of preferably 300 mesh to 400 mesh, more preferably 300 mesh to 350 mesh. In the present disclosure, the drying is conducted at preferably 30° C. to 60° C., more preferably 40° C. to 50° C.; there is no special limit to a drying time herein, as long as the moisture content is not more than 10%. In the present disclosure, after the drying, the dried particles are preferably packaged to obtain the FPG composite particle. There is no special limitation on the packaging herein, and packaging methods well known to those skilled in the art can be used.

The present disclosure provides an FPG composite lozenge, including a core tablet and a coating layer coated on a surface of the core tablet. In the present disclosure, the core tablet includes preferably the following components in parts by mass: 65 parts to 85 parts, preferably 70 parts to 80 parts, and more preferably 75 parts of the FPG; 5 parts to 15 parts, preferably 8 parts to 12 parts, and more preferably 10 parts of maltodextrin; 5 parts to 10 parts, preferably 6 parts to 9 parts, and more preferably 7 parts to 8 parts of fructooligosaccharide; 3 parts to 8 parts, preferably 4 parts to 7 parts, and more preferably 5 parts to 6 parts of erythritol; 5 parts to 8 parts, preferably 5.5 parts to 7.5 parts, and more preferably 6 parts to 7 parts of calcium carbonate; and 2 parts to 5 parts, preferably 2.5 parts to 4.5 parts, and more preferably 3 parts to 4 parts of a binder. In the present disclosure, the binder preferably includes hydroxypropyl methylcellulose (HPMC) and/or gum arabic. In the present disclosure, the FPG composite lozenge is preferably taken orally, with a dosage of preferably 2 to 3 tablets/d; the FPG composite lozenge has a single-tablet mass of preferably 0.4 to 0.6 g/tablet, more preferably 0.5 to 0.6 g/tablet.

In the present disclosure, a preparation method of the FPG composite lozenge preferably includes the following steps: mixing the FPG, maltodextrin, fructooligosaccharide, erythritol, calcium carbonate, and HPMC to allow granulation, drying, and tabletting in sequence to obtain the FPG composite lozenge. In the present disclosure, the raw materials for preparing the FPG lozenge are preferably superfine grinded and then screened before use. There is no particular limitation on the superfine grinding herein, as long as the raw materials can be grinted to a mesh size of no less than 300 mesh. A sieve mesh has a size of preferably 300 mesh, and a resulting undersize is collected. In the present disclosure, the granulation is preferably conducted in a boiling granulator or a spray granulator; and granules obtained by the granulation have a particle size of preferably 300 mesh to 350 mesh, more preferably 300 mesh. In the present disclosure, the drying is conducted at preferably 30° C. to 100° C., more preferably 50° C. to 80° C.; there is no special limit to a drying time herein, as long as the moisture content is not more than 10%. In the present disclosure, after the tableting is completed, an obtained tablet material is preferably packaged to obtain the FPG composite lozenge. There is no special limitation on the packaging herein, and packaging methods well known to those skilled in the art can be used.

The technical solution provided by the present disclosure will be described in detail below with reference to the examples, but they should not be construed as limiting the claimed scope of the present disclosure.

### Example 1

A Flammulina velutipes dry powder was subjected to hot water extraction at 90° C. for 4 h, and a resulting water extract was subjected to protein removal using a Sevag reagent to obtain a protein-free water extract. The protein-free water extract was subjected to alcohol precipitation with absolute ethanol at 4° C. for 48 h, and a resulting PG precipitate was subjected to first dialysis using a 3 kDa dialysis bag for 24 h, and then freeze-dried at −60° C. for 72 h to obtain a crude PG; the crude PG was dissolved in water, and then sequentially subjected to separation and purification by DEAE Sepharose Fast Flow and Sephadex G-200 chromatography (separation range: 5 kDa to 600 kDa) with ultrapure water (flow rate: 1 mL/min) as an eluent, an eluate was collected with absorption peaks appearing simultaneously at the wavelengths of 480 nm and 295 nm; the eluate was subjected to second dialysis using a 3 kDa dialysis bag for 24 h, and then freeze-dried at −60° C. for 72 h to obtain the FPG. The Flammulina velutipes dry powder and the hot water were at a solid-to-liquid ratio of 1:20 (1 g:20 mL), the protein-free water extract and the absolute ethanol were at a volume ratio of 1:4, and the crude PG and the water for dissolving were at a material-to-water ratio of 3 g:10 mL.

The FPG had a yield of 79.32%, a purity of 95.6%, and a molecular weight of FPG of 33.05 kDa; the glucose, D-galactose, and xylose were at a molar ratio of 24:4:1; and a peptide chain end and the polysaccharide chain were ligated by O-glycosidic bonds. Yield=(mass of FPG/mass of Flammulina velutipes dry powder)×100%; the purity was determined using a TSK-gel PWXL G4000 column (7.8 mm×300 mm, 5 μm) equipped with an evaporative light scattering detector (ELSD) coupled with an Agilent 1200 series HPLC instrument.

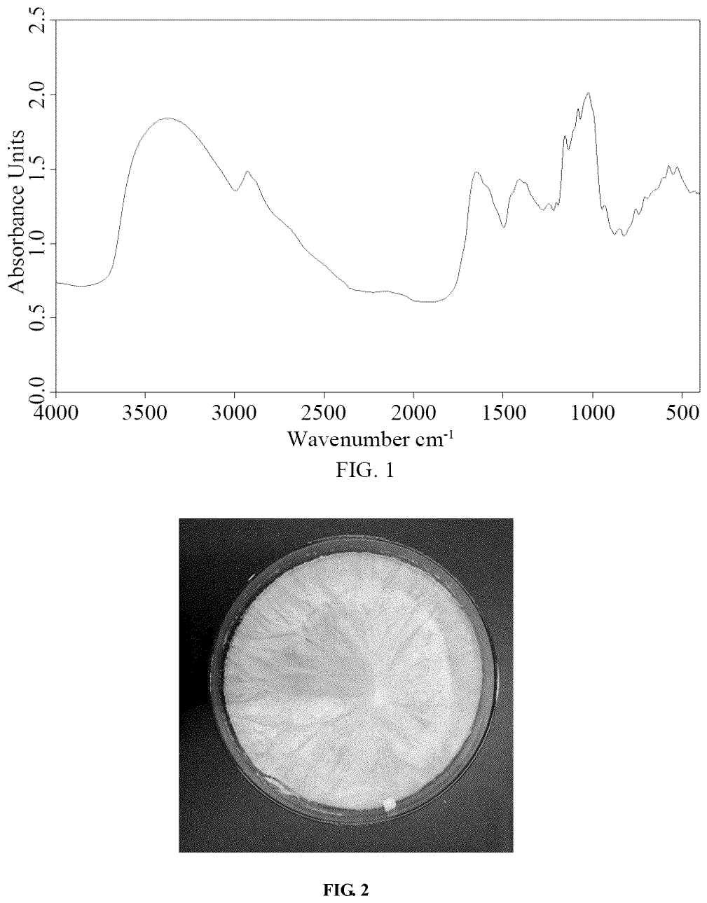

FIG. 1 showed an infrared spectrum of the FPG prepared in Example 1. As shown in FIG. 1, the FPG had a peak at 3,000 cm−1 (stretching vibration peak of hydroxyl groups) and an absorption peak (stretching vibration peak of C—C) near 1,295 cm−1, and was preliminarily determined to be a carbohydrate compound. The absorption peak of the sample near 1,630 cm−1 was caused by the asymmetric stretching vibration of the carbonyl group in the acetyl group. This peak was a characteristic absorption peak of protein substances, indicating that the sample contained protein groups. A relatively strong absorption peak near 1,080 cm−1 was caused by the stretching vibration of pyranose, indicating that the sugar chain configuration was a pyran ring structure. In addition, the infrared spectrum of PG showed that there was a protruding peak at 890 cm−1, which corresponded to the absorption peak of D-glucan. This indicated that the sample contained D-glucan, and the infrared spectrum could determine that the sample was PG.

FIG. 2 showed a physical map of the FPG prepared in Example 1. As shown in FIG. 2, the FPG obtained using the dual-wavelength screening method provided by the present disclosure appeared a light color, indicating that the dual-wavelength screening method provided by the present disclosure could significantly improve the purity of FPG.

### Example 2

The FPG was prepared according to the method of Example 1, and the only difference from Example 1 was that: the solid-liquid ratio was 1:30, the protein-free water extract and the absolute ethanol were at a volume ratio of 1:3.5, the crude PG and the water for dissolving were at a material-to-water ratio of 3 g:10 mL, and the dialysis bag for the first dialysis had a pore size of 5 kDa.

The FPG had a yield of 75.17%, a purity of 90.3%, and a molecular weight of FPG of 30.42 kDa; the glucose, D-galactose, and xylose were at a molar ratio of 25:2:6; and a peptide chain end and the polysaccharide chain were ligated by O-glycosidic bonds.

### Comparative Example 1

The FPG was prepared according to the method of Example 1, and the only difference from Example 1 was that: the dialysis bag for the first dialysis had a pore size of 5 kDa, and an eluate with the absorption peak appearing at 480 nm in a phenol-sulfuric acid method was collected.

The FPG had a yield of 83.88%, a purity of 78.61%.

### Comparative Example 2

The novel proteoglycan PGD1-1 disclosed in the literature “Isolation, characterization and HepG-2 inhibition of a novel proteoglycan from Flammulina velutipes. International Journal of Biological Macromolecules 189 (2021) 11-17”.

### Test Example 1

### Inhibition Rate Test on HepG-2 (Human Liver Cancer Cells)

The inhibitory effect of FPG samples prepared in Examples 1 to 2 and Comparative Examples 1 to 2 on the viability of HepG-2 cells was measured by the MTT method, the specific steps are as follows: fluorouracil (5-Fu) was used as a positive control group. The HepG-2 cells were pre-cultured, and the HepG-2 cells growing on a 96-well cell culture plate were observed under an inverted biological microscope. After adhesion of cells, the upper medium was removed, and 100 μL of medium containing different groups of PG (200 μg/mL) was added, and 100 μL of HepG-2 cells were inoculated in the 96-well plate at 1×105 cells/mL. The 5-Fu (50 μg/mL) group was set as a positive control group and 100 μL of MTT (0.5 mg/mL) was added. After incubation for 4 h in a constant-temperature incubator (37° C., 5% CO2), the medium was removed and DMSO (150 μL) was added into each well. 5 parallel experiments were set up for each group of samples. The absorbance at 550 nm was measured by a microplate reader.

The results of the inhibitory effect of FPG prepared in Examples 1 to 2 and Comparative Example 1 on the viability of HepG-2 cells were shown in Table 1.

TABLE 1
Inhibitory effects of different concentrations
of FPG on HepG-2 cell viability

| | Inhibition rate of different concentrations of FPG/% | | | |
|---|---|---|---|---|
| FPG | 25 μg/mL | 50 μg/mL | 100 μg/mL | 200 μg/mL |
| Example1 | 37.85 | 39.11 | 41.51 | 45.02 |
| Example2 | 35.63 | 37.03 | 37.62 | 41.29 |
| Comparative | 29.87 | 35.01 | 36.15 | 38.76 |
| Example1 | | | | |
| Comparative | 31.23 | 36.12 | 36.72 | 37.96 |
| Example 2 | | | | |

As shown in Table 1, the FPG obtained at both 480 nm and 295 nm using the dual-wavelength screening method provided by the present disclosure had both the absorption wavelength of polysaccharide and the absorption wavelength of protein, and showed anti-liver cancer activity.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A dual-wavelength screening method of a Flammulina velutipes proteoglycan (FPG), comprising the following steps: subjecting Flammulina velutipes to water extraction and protein removal in sequence to obtain a protein-free water extract; mixing the protein-free water extract with absolute ethanol to allow alcohol precipitation to obtain a proteoglycan (PG) precipitate; wherein the protein-free water extract and the absolute ethanol are at a volume ratio of 1:3.5 to 1:4; dialyzing the PG precipitate to obtain a crude PG; wherein the dialyzing is conducted at a molecular weight cut-off of 3 kDa to 5 kDa; and subjecting the crude PG to separation by Sepharose FF ion exchange chromatography and Sephadex G-200 chromatography in sequence using water as an eluent, and collecting a component with absorption peaks at both 480 nm and 295 nm to obtain the FPG.

2. The dual-wavelength screening method according to claim 1, wherein a dry weight of the Flammulina velutipes and a volume of water for the water extraction are at a ratio of 1 g:20 to 1 g:30 mL; and the water extraction is conducted at 85° C. to 95° C. for 3.5 h to 4.5 h.

3. The dual-wavelength screening method according to claim 1, wherein the alcohol precipitation is conducted at 0° C. to 25° C. for 24 h to 72 h.

4. The dual-wavelength screening method according to claim 1, wherein the dialyzing is conducted for 20 h to 28 h.

5. The dual-wavelength screening method according to claim 1, wherein the crude PG is dissolved in water to allow the separation by the Sepharose FF ion exchange chromatography; and a dry weight of the crude PG and a volume of the water are at a ratio of 1 g:1 to 1:15 mL.

6. An FPG prepared by the dual-wavelength screening method according to claim 1, wherein a monosaccharide unit of the FPG comprises glucose, D-galactose, and xylose; and a peptide chain end of the FPG is ligated to a polysaccharide chain by an O-glycosidic bond.

7. An anti-liver cancer drug, comprising the FPG according to claim 6 and a pharmaceutically acceptable excipient.

8. An FPG composite particle, comprising the following components in parts by mass: 65 parts to 85 parts of the FPG according to claim 6, 5 parts to 15 parts of maltodextrin, 5 parts to 10 parts of fructooligosaccharide, 3 parts to 8 parts of erythritol, 5 parts to 8 parts of calcium carbonate, and 2 parts to 5 parts of a binder.

9. An FPG composite lozenge, comprising a core tablet and a coating layer coated on a surface of the core tablet; wherein the core tablet comprises the following components in parts by mass: 65 parts to 85 parts of the FPG according to claim 6, 5 parts to 15 parts of maltodextrin, 5 parts to 10 parts of fructooligosaccharide, 3 parts to 8 parts of erythritol, 5 parts to 8 parts of calcium carbonate, and 2 parts to 5 parts of a binder.

10. The dual-wavelength screening method according to claim 3, wherein a dry weight of the Flammulina velutipes and a volume of water for the water extraction are at a ratio of 1 g:20 to 1 g:30 mL; and the water extraction is conducted at 85° C. to 95° C. for 3.5 h to 4.5 h.
